(19) United States
(12) Patent Application Publication
(10) Pub. No.: US 2024/0246917 A1
(43) Pub. Date: Jul. 25, 2024

(54) PROCESS FOR PREPARING A CDK INHIBITOR

(71) Applicant: Aurigene Oncology Limited, Bangalore (IN)

(72) Inventors: Ramulu PODDUTOORI, Telangana Karimnagar (IN); Uday Vijaykumar BHAT, Bangalore (IN); Devaraja Thimmasandra Seethappa, Chikkaballapura (D), Karnataka (IN)

(21) Appl. No.: 18/288,498

(22) PCT Filed: Apr. 26, 2022
(86) PCT No.: PCT/IB2022/053851

§ 371 Date: Oct. 26, 2023

(30) Foreign Application Priority Data
Apr. 27, 2021 (IN) 202141019263

(51) Int. Cl. C07D 231/40; C07C 25/02

(52) U.S. Cl. CPC C07D 231/40; C07C 25/02

(57) ABSTRACT

The present invention relates to the preparation of a CDK7 inhibitor having the structure of compound of formula (I). The invention described herein also relates to intermediates useful for preparing compound of formula (I) and methods of preparation of those intermediates.

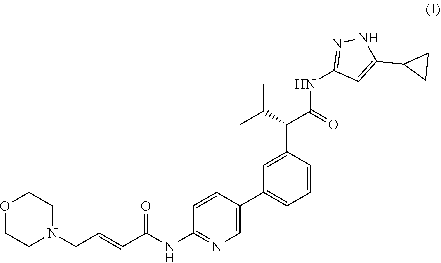

### RELATED APPLICATION

This application claims the benefit of Indian provisional application number 202141019263, filed on 27 Apr. 2021, the contents of which are hereby incorporated by reference in its entirety.

### FIELD OF THE INVENTION

The present invention is directed to methods of preparation of compound of formula (I) that is useful for inhibiting Cyclin-dependent kinase 7 (CDK7) and for treating diseases or disorders mediated thereby. The present invention also relates to intermediates and methods of their preparation that are useful in the preparation of compound of formula (I).

### BACKGROUND OF THE INVENTION

CDK7, which complexes with cyclin H and RING-finger protein MATI, phosphorylates the cell cycle CDKs in the activation of T-loop, to promote their activities (Fisher et al., Cell., August 26;78(4):713-24, 1994). As such, it has been proposed that inhibiting CDK7 would provide a potent means of inhibiting cell cycle progression, which may be especially relevant given that there is compelling evidence from gene knockout studies in mice for lack of an absolute requirement for CDK2, CDK4 and CDK6 for the cell cycle at least in most cell types (Malumbres et al., Nature Cell Biology, 11, 1275-1276, 2009), whilst different tumors appear to require some, but they are independent of other interphase CDKs (CDK2, CDK4, CDK6). Recent genetic and biochemical studies have confirmed the importance of CDK7 for cell cycle progression (Larochelle. et al., Mol Cell., March 23;25(6):839-50. 2007; Ganuza et al., EM BO J., May 30; 31(11): 2498-510, 2012).

Cyclin-dependent kinase 7 (CDK7) activates cell cycle CDKs and is a member of the general Transcription factor II Human (TFIIH). CDK7 also plays a role in transcription and possibly in DNA repair. The trimeric Cak complex CDK7/CyclinH/MATI is also a component of TFIIH, the general transcription/DNA repair factor IIH (Morgan, DO., Annu.Rev. Cell Dev. Biol. 13, 261-91, 1997). As a TFIIH subunit, CDK7 phosphorylates the CTD (Carboxy-Terminal-Domain) of the largest subunit of RNA polymerase II (pol II). The CTD of mammalian pol (II) consists of 52 heptad repeats with the consensus sequence ¹YSPTSPS⁷ and the phosphorylation status of the Ser residues at positions 2 and 5 has been shown to be important in the activation of RNAP-II indicating that it is likely to have a crucial role in the function of the CTD. CDK7, which primarily phosphorylates Ser-5 (PSS) of RNAP-II at the promoter as part of transcriptional initiation (Gomes et al., Genes Dev. 2006 Mar 1; 20(5):601-12, 2006), in contrast with CDK9, which phosphorylates both Ser-2 and Ser-5 of the CTD heptad (Pinhero et al., Eur. J. Biochem., 271, pp. 1004-1014, 2004).

In addition to CDK7, other CDKs have been reported to phosphorylate and regulate RNA pol (II) CTD. The other CDKs include, Cdk9/ Cyclin T1 or T2 that constitute the active form of the positive transcription elongation factor (P-TEFb) (Peterlin and Price, Mol Cell., August 4; 23(3): 297-305,2006) and Cdk12/Cyclin K and Cdk13/Cyclin K as the latest members of RNAPII CTD kinases (Bartkowiak et al., Genes Dev., Oct 1 5;24(20):2303-16, 2010; Blazek et al., Genes Dev. Oct 15;25(20):2158-72, 2011).

Disruption of RNAP II CTD phosphorylation has been shown to preferentially effect proteins with short half-lives, including those of the anti-apoptotic BCL-2 family. (Konig et al., Blood, 1, 4307-4312, 1997; The transcriptional non-selective cyclin-dependent kinase inhibitor flavopiridol induces apoptosis in multiple myeloma cells through transcriptional repression and down-regulation of Mcl-1; (Gojoet al., Clin. Cancer Res. 8, 3527-3538, 2002).

This suggests that the CDK7 enzyme complexes are involved in multiple functions in the cell: cell cycle control, transcription regulation and DNA repair. It is surprising to find one kinase involved in such diverse cellular processes, some of which are even mutually exclusive. It also is puzzling that multiple attempts to find cell cycle dependent changes in CDK7 kinase activity remained unsuccessful. This is unexpected since activity and phosphorylation state of its substrate, CDC2, fluctuate during the cell cycle. In fact, it is shown that cdk7 activity is required for the activation of both Cdc2/Cyclin A and Cdc2/Cyclin B complexes, and for cell division. (Larochelle, S. et al. Genes Dev 12,370-81, 1998). Indeed, flavopiridol, a non- selective pan-CDK inhibitor that targets CTD kinases, has demonstrated efficacy for the treatment of chronic lymphocytic leukemia (CLL), but suffers from a poor toxicity profile (Lin et al.,]. Clin. Oncol.27, 6012-6018, 2009; Christian et al., Clin. Lymphoma Myeloma,9, Suppl. 3, S179-S185, 2009).

International publication WO2016193939, which is incorporated herein by reference for all purposes describes CDK7 inhibitors and processes for the preparation thereof. Inhibitors of CDK7 are currently being developed for the treatment of cancer. For drug development, it is typically advantageous to employ individual stereoisomers as they exhibit marked differences in pharmacodynamic, pharmacokinetic, and toxicological properties.

Accordingly, there is a need for an improved process for the preparation of specific stereoisomers of CDK7-inhibiting molecules for suitable pharmacological effects on any individual.

### SUMMARY OF THE INVENTION

Compound of formula (I) is also known as (S, E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide. The present disclosure provides processes and synthesis of the compound of formula (I) with high yield and purity. The present disclosure also discloses certain intermediates obtained in the process of preparing compound of formula (I).

In one aspect, present invention provides a method for preparing a compound of formula (I):

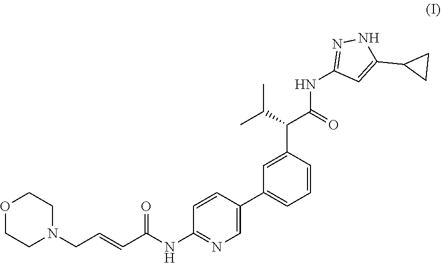

the method comprising: reacting compound of formula (I′″):

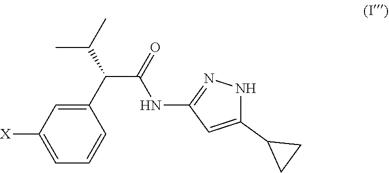

with a compound of formula (KRM-C1)

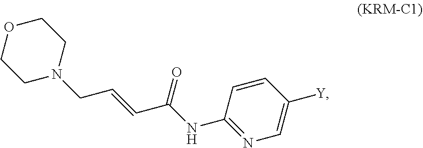

wherein X is Br, Cl or I; Y is —B(OH)₂ or

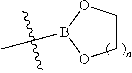

optionally substituted with 1, 2, 3 or 4 independently selected C₁₋₄ alkyl substituents, wherein the subscript n is 1 or 2.

In another aspect, the present invention provides a method for preparing a compound of formula (I):

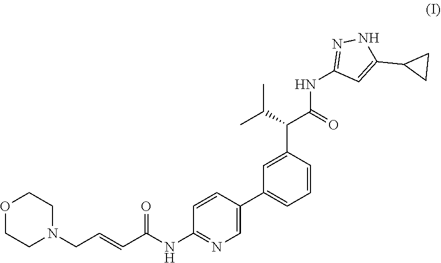

wherein the method comprising:
i. reacting a compound of formula (1)

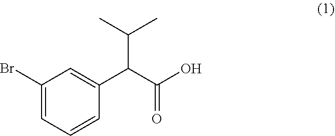

with a compound of formula (2)

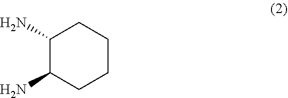

to afford a salt of formula (3)

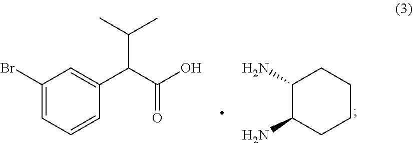

ii. recrystallizing the salt of formula (3) in a solvent (B) to obtain a compound of formula

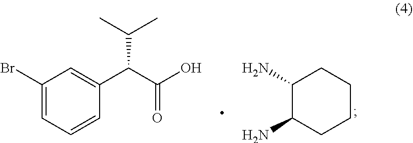

iii. treating a mixture comprising the salt of formula (4) and a solvent (C) with an acid to generate a compound of formula (KRM-A)

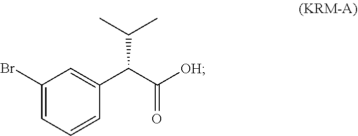

iv. reacting the compound of formula (KRM-A) with a compound of formula (KRM-B)

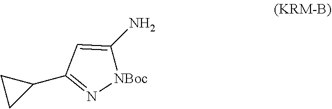 [SMILES: CC(C)(C)OC(=O)n1nc(C2CC2)cc1N]

to afford a compound of formula (I′):

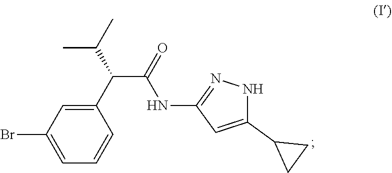 [SMILES: CC(C)[C@H](C(=O)Nc1cc(C2CC2)[nH]n1)c1cccc(Br)c1]

and
v. reacting the compound of formula (I′) with a compound of formula (KRM-C)

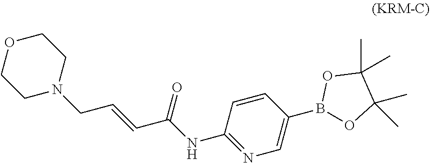 [SMILES: CC1(C)OB(c2ccc(NC(=O)/C=C/CN3CCOCC3)nc2)OC1(C)C]

to obtain the compound of formula (I).

In another aspect, the present invention provides a compound of formula (III):

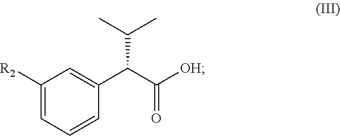

wherein
R² is halo, methylsulfonyloxy, p-tolylsulfonyloxy, phenylsulfonyloxy or trifluoromethylsulfonyloxy.

In another aspect, the present invention provides a compound of formula (IV):

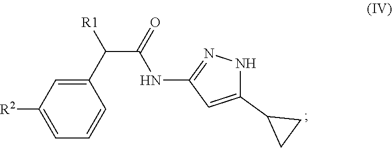

wherein
R¹ is C₁₋₆ alkyl; and R² is halo, methylsulfonyloxy, p-tolylsulfonyloxy, phenylsulfonyloxy or trifluoromethylsulfonyloxy.

In further aspect, the present invention relates to methods of preparation of compound of formula (KRM-A).

### DETAILED DESCRIPTION OF THE INVENTION

As used in the present specification, the following words and phrases are generally intended to have the meanings as set forth below, except to the extent that the context in which they are used indicates otherwise.

As used herein, the term “alkyl” refers to a branched or straight hydrocarbon chain of one to ten carbon atoms, such as methyl, ethyl, n-propyl, isopropyl, n-butyl, isobutyl, sec-butyl, t-butyl, pentyl, hexyl, heptyl, octyl, and the like. The term “Cₙ₋ₘ alkyl” or (Cₙ-Cₘ) alkyl, refers to an alkyl group having n to m carbon atoms. C₁₋₆ alkyl is preferred.

As used herein, the term “halo” or “halogen” alone or in combination with other term(s) means fluoro, chloro, bromo or iodo.

As used herein, the term “about” when referring to a number or a numerical range means that the number or numerical range referred to, is an approximation within experimental variability (or within statistical experimental error), and thus the number or numerical range may vary from, for example, between 1% and 15% of the stated number or numerical range.

It is to be understood by one of ordinary skill in the art that the present discussion is a description of exemplary embodiments only and is not to be construed as limiting the broader aspects of the present invention. In fact, it will be apparent to those skilled in the art that various modifications and variations can be made to the compounds, compositions, and methods described herein without departing from the scope or spirit of the invention. For instance, features illustrated or described as part of one embodiment can be applied to another embodiment to yield a still further embodiment. Thus, it is intended that the present invention include such modifications and variations and their equivalents. Other objects, features, and aspects of the present invention are disclosed in, or are obvious from, the following detailed description.

In one embodiment, the present invention provides a method for preparing a compound of formula (I):

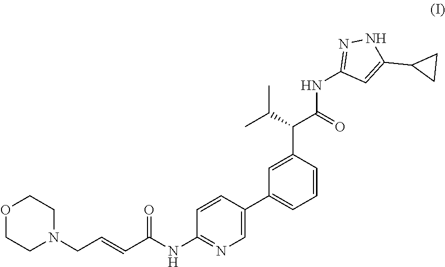 [SMILES: CC(C)[C@H](C(=O)Nc1cc(C2CC2)[nH]n1)c1cccc(-c2ccc(NC(=O)/C=C/CN3CCOCC3)nc2)c1]

wherein the method comprising: reacting a compound of formula (I′″):

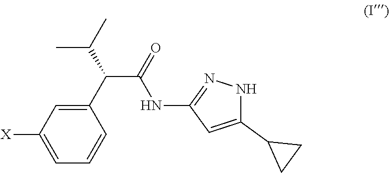

with a compound of formula (KRM-C1)

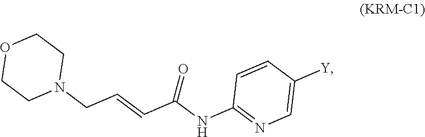

wherein X is Br, Cl or I; Y is —B(OH)₂ or

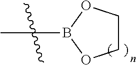

optionally substituted with 1, 2, 3 or 4 independently selected C₁₋₄ alkyl substituents, wherein the subscript n is 1 or 2.

In one embodiment, the reaction of the compound of formula (I′″) with the compound of formula (KRM-C1) is carried out in the presence of a palladium catalyst.

In one embodiment, the compound of formula (I′″) is prepared by reacting a compound of formula (KRM-A1):

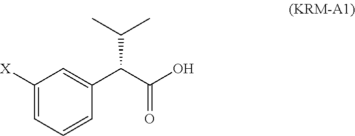

with a compound of formula (KRM-B):

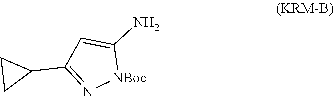

wherein X is Br, Cl or I.

In one embodiment, the compound of formula (KRM-A1) is prepared by a method comprising of:
a) reacting a compound of formula (KRM-A2):

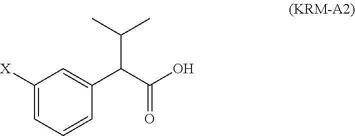

with (1R,2R)-cyclohexane-1,2-diamine to form a salt having a formula (5):

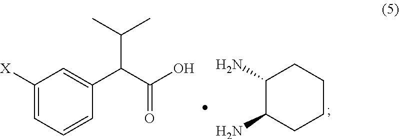

b) recrystallizing the salt compound of formula (5) to obtain salt (6) having a formula (6):

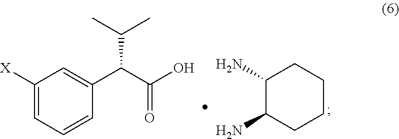

and
c) treating the salt of formula (6) with an acid to obtain the compound of formula (KRM-A1), wherein X is Br, Cl or I.

In one embodiment, the step of recrystallization is carried out in acetone, acetonitrile, methanol, isopropyl alcohol, isopropyl acetate, isobutanol, 1-pentanol, 1-propanol, ethanol, water, or a mixture thereof.

In one embodiment, wherein the acid is HCl. In one embodiment, X is Br.

In one embodiment, Y is —B(OH)₂.

In one embodiment, Y is

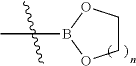

optionally substituted with 1, 2, 3 or 4 independently selected C₁₋₄ alkyl substituents, wherein the subscript n is 1 or 2. In one embodiment, Y is

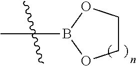

substituted with at least four C₁₋₄ alkyl substituents and the subscript n is 1.

In one embodiment, Y is

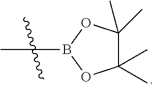 [SMILES: *B1OC(C)(C)C(C)(C)O1]

In one embodiment, the present invention provides a method for preparing a compound of formula (I):

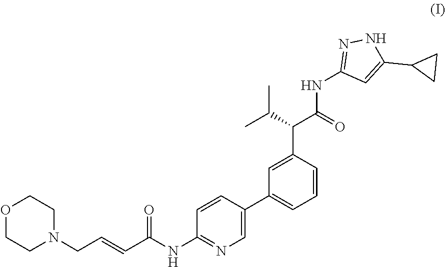 [SMILES: CC(C)[C@H](C(=O)Nc1cc(C2CC2)[nH]n1)c1cccc(-c2ccc(NC(=O)/C=C/CN3CCOCC3)nc2)c1]

the method comprising:
i. reacting a compound of formula (1)

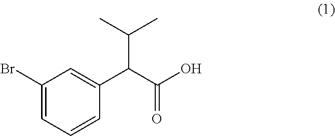

with a compound of formula (2)

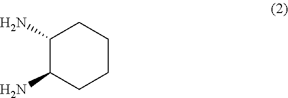

to afford a salt of a formula (3)

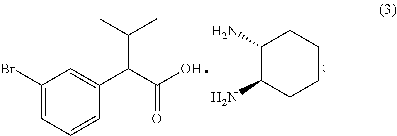

ii. recrystallizing the salt of formula (3) in a solvent (B) to obtain a compound of formula

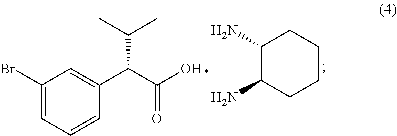

iii. treating a mixture comprising compound of formula (4) and a solvent (C) with an acid to generate a compound of formula (KRM-A)

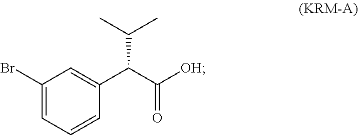

iv. reacting a compound of formula (KRM-A) with a compound of formula (KRM-B)

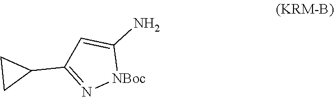 [SMILES: CC(C)(C)OC(=O)n1nc(C2CC2)cc1N]

to afford a compound of formula (I′):

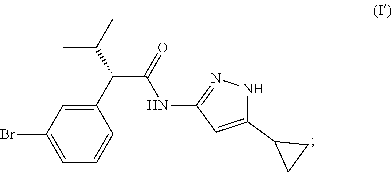 [SMILES: CC(C)[C@H](C(=O)Nc1cc(C2CC2)[nH]n1)c1cccc(Br)c1]

and
v. reacting the compound of formula (I′) with a compound of formula (KRM-C)

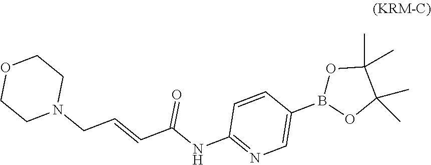 [SMILES: CC1(C)OB(c2ccc(NC(=O)/C=C/CN3CCOCC3)nc2)OC1(C)C]

to obtain the compound of formula (I).

In one embodiment, the present invention provides a method for preparing a compound of formula (KRM-A), wherein the method comprises:
A. reacting the compound of formula (1) with the compound of formula (2) to afford a salt of formula (3)

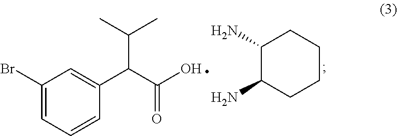

B. recrystallizing the salt of formula (3) in the solvent (B) to obtain the compound of formula (4)

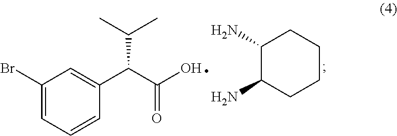

and
C. treating a mixture comprising the salt of formula (4) and solvent (C) with the acid to generate compound of formula (KRM-A).

In one embodiment, step i) is carried out in the presence of the solvent (A) selected from acetone, dichloromethane, n-propyl acetate, acetonitrile, methanol, isopropyl alcohol, isopropyl acetate, isobutanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, solvent (A) is acetonitrile, isopropyl alcohol, isopropyl acetate, water or mixtures thereof.

In one embodiment, step i) is carried out at a temperature between about 60° C.to about 100° C.

In one embodiment, step i) comprises reacting the compound of formula (1) with the compound of formula (2) in the presence of acetonitrile or mixture of IPA and water, heating the reaction mixture to about 100° C. and cooling the reaction mixture to ambient temperature. In one embodiment, step i) further comprises the filtering of compound of formula (3) from the reaction mixture.

In one embodiment, step ii) comprises:
a) providing a mixture comprising the compound of formula (3) and the solvent (B);
b) heating the mixture to form a solution; and
c) bringing the solution to supersaturation thereby causing the compound of formula (4) to precipitate out of the solution.

In one embodiment, solvent (B) is acetone, acetonitrile, methanol, isopropyl alcohol, isopropyl acetate, isobutanol, 1-pentanol, 1-propanol, ethanol, water, or mixtures thereof. In one embodiment, solvent (B) is isopropyl alcohol or water or any mixtures thereof.

In one embodiment, in step ii), the reaction mixture was refluxed. In one embodiment, the reaction mixture was refluxed at a temperature between about 65° C. to about 100° C. In one embodiment, the reaction mixture was refluxed at about 100° C.and then cooled to ambient temperature.

In one embodiment, the step of bringing the solution to supersaturation comprises cooling the solution to ambient temperature or lower.

In one embodiment, the step of bringing the solution to supersaturation comprises maintaining a solution temperature above about 20° C.

In one embodiment, step ii) further comprises filtering the compound of formula (4) from the mixture comprising compound of formula (4).

In one embodiment, steps a) to c) of step ii) are repeated at least three times.

In one embodiment, the mixture comprising compound of formula (4) and a solvent (C) in step iii) is a suspension.

In one embodiment, step iii) comprises cooling the mixture comprising compound of formula (4) and a solvent (C).

In one embodiment, solvent (C) is acetone, dichloromethane, n-propyl acetate, acetonitrile, methanol, isopropyl acetate, isobutanol, 2-butanol, 1-butanol, n-butyl acetate, 1-pentanol, 1-propanol, chloroform, methyl acetate, isobutyl acetate, isobutanol or ethanol.

In one embodiment, the acid is HCl.

In one embodiment, method of preparing a compound of (KRM-A) according to steps i) to iii) further comprising isolating the compound of formula (KRM-A) from the mixture. In one embodiment, the mixture is a solution and isolating the compound of formula (KRM-A) comprises filtering the compound of formula (KRM-A) from the mixture.

In one embodiment, the compound of formula (II′) is prepared by i) reacting the compound of formula (KRM-A) with oxalyl chloride to generate a compound of formula

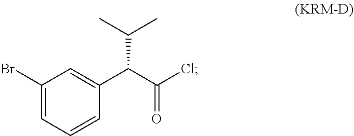

ii) reacting the compound of formula (KRM-D) in situ with the compound of formula (KRM-B).

In one embodiment, in step i) of the preparation of compound of formula (II′), oxalyl chloride is added at 0° C. to the reaction mixture comprising compound of formula (KRM-A) and a solvent selected from dry DCM and DMF, or mixtures thereof; and the mixture is allowed to attain ambient temperature.

In one embodiment, step i) of the preparation of compound of formula (II′), further comprises isolating compound of formula (KRM-D) from the mixture comprising compound of formula (KRM-D). In one embodiment, compound of formula (KRM-D) is isolated by concentrating the reaction mixture under vacuum. In one embodiment, concentrating the reaction mixture is carried out at a temperature between about 40° C. and about 45° C.

In one embodiment, step ii) of the preparation of compound of formula (II′) comprises: ii-a) providing a mixture comprising compound of formula (KRM-D) and toluene; ii-b) reacting the said mixture with a pre-cooled solution of compound of formula (KRM-B) and a base to afford compound of formula (II′). In one embodiment, step ii) of the preparation of compound of formula (II′) is carried out in the presence of a base. In one embodiment, the base is N,N-diisopropylethylamine.

In one embodiment, method of preparing compound of formula (II′), further comprises isolation of compound of formula (II′) from the mixture comprising compound of formula (II′). vacuum.

In one embodiment, compound of formula (1) is prepared by reacting a compound of formula (X_{A})

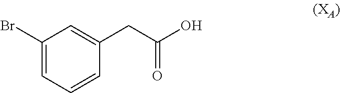 [SMILES: O=C(O)Cc1cccc(Br)c1]

with isopropyl bromide (X_{B}) in the presence of a base and a solvent. In one embodiment, the base is lithium diisopropylamide (LDA), and the solvent is THF. In one embodiment, the method of preparing compound of formula (1) further comprises, isolating the compound of formula (1) from the mixture comprising compound of formula (1).

In one embodiment, the compound of formula (1) is prepared by: reacting a compound of formula (X_{A})

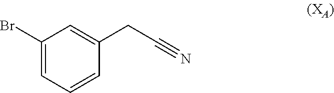

with CH₃CH(Br)CH₃ (X_{B}) in the presence of a base to obtain a compound of formula (X_{C})

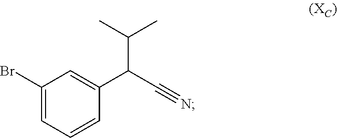

and reacting the compound of formula (X_{C}) in the presence of an acid followed by reflux.

In one embodiment, the base is KOH and the reaction is carried out in the presence of quaternary ammonium salt. In one embodiment, quaternary ammonium salt is tetrabutylammonium bromide. In one embodiment, the acid is sulfuric acid and the reflux is carried out at 140° C. for 12 h. In one embodiment, the method of preparing compound of formula (1) further comprises, isolating the compound of formula (1) from the mixture comprising compound of formula (1).

In one embodiment, compound of formula (I′) is prepared by reacting a compound of formula (II′):

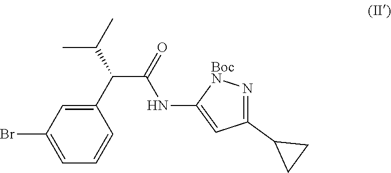 [SMILES: CC(C)[C@H](C(=O)Nc1cc(C2CC2)nn1C(=O)OC(C)(C)C)c1cccc(Br)c1]

with a deprotecting agent.

In one embodiment, the deprotecting agent is HCl.

In one embodiment, the method of preparing a compound of formula (I′) further comprises, isolating the compound of formula (I′) from the mixture comprising compound of formula (I′).

In one embodiment of preparing a compound of formula (I), step v) is carried out in the presence of a palladium catalyst. In one embodiment of preparing a compound of formula (I), step v) is carried out in the presence of a solvent. In one embodiment of preparing a compound of formula (I), the solvent is a mixture of dioxane and water.

In one embodiment, the method of preparing compound of formula (I) comprises, isolating the compound of formula (I) from the mixture comprising compound of formula (I).

In one embodiment, the mixture comprises a solution of the compound of formula (I). In one embodiment, the solution comprises a solid crude material comprising the compound of formula (I) dissolved in a solvent. In one embodiment, a solid crude material comprises about 70% to about 90% compound of formula (I).

In one embodiment, the step of isolating the compound of formula (I) from the mixture comprises: filtering, washing and drying the compound of formula (I) obtained from the mixture. In one embodiment, the compound of formula (I) filtered from the mixture is washed with a solvent.

In one embodiment, the method of preparing compound of formula (I) further comprises purification of solid crude material comprising the compound of formula (I).

In one embodiment, the present invention provides a compound of formula (III):

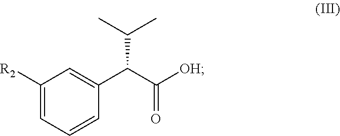

wherein
R² is halo, methylsulfonyloxy, p-tolylsulfonyloxy, phenylsulfonyloxy or trifluoromethylsulfonyloxy.

In one embodiment of compound of formula (III), R² is halo.

In one embodiment, compound of formula (III) is a compound having the structure of

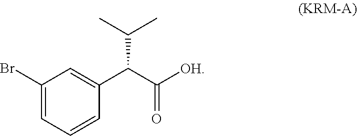

In one embodiment, the present invention provides a compound of formula (IV):

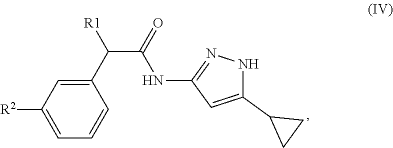

wherein:
R¹ is C₁₋₆ alkyl; and R² is halo, methylsulfonyloxy, p-tolylsulfonyloxy, phenylsulfonyloxy or trifluoromethylsulfonyloxy.

In one embodiment, the present invention provides a compound of formula (IV):

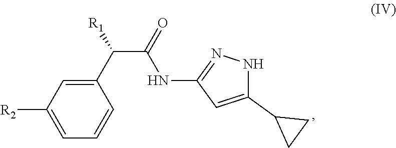

wherein: R¹ is C₁₋₆ alkyl; and R² is halo, methylsulfonyloxy, p-tolylsulfonyloxy, phenylsulfonyloxy or trifluoromethylsulfonyloxy.

In one embodiment of compound of formula (IV), R² is halo.

In one embodiment, the compound of formula (IV) is a compound having the structure:

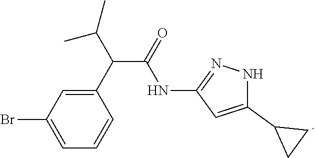 [SMILES: CC(C)C(C(=O)Nc1cc(C2CC2)[nH]n1)c1cccc(Br)c1]

In one embodiment, the compound of formula (IV) is a compound having the structure

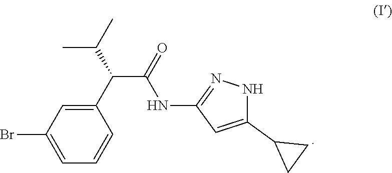 [SMILES: CC(C)[C@H](C(=O)Nc1cc(C2CC2)[nH]n1)c1cccc(Br)c1]

### Abbreviations

The following abbreviations are used in this specification:

ACN—Acetonitrile; IPA—Isopropyl alcohol; HCl—Hydrochloric acid; TFA—Trifluoro acetic acid; EtOAc—Ethyl Acetate; conc—Concentrated; CHCl₃—Chloroform; CDCl₃//chloroform-d—Deuterated Chloroform; DMSO-d₆—Deuterated dimethylsulfoxide; DCM—Dichloromethane; DMF—N,N-Dimethylformamide; g—Gram; h—Hours; ¹H—Proton; J—Coupling Constant; HPLC—High-performance liquid chromatography; chiral HPLC—chiral high-performance liquid chromatography; LDA—Lithium diisopropylamide; M—Molar; MHz—Mega Hertz (frequency); MS—Mass Spectroscopy; mmol—Milli mole; mL—Milli litre; min—Minutes; mol—Moles; M⁺—Molecular ion; m/z—mass to charge ratio; Na₂SO₄—Sodium sulfate; N—Normality; NMR—Nuclear Magnetic Resonance; Pd(dppf)Cl₂·DCM—[1,1′-Bis(diphenylphosphino)ferrocene]dichloropalladium(II) complex with dichloromethane; rt/RT—Room temperature or ambient temperature ranging between 22° C.-25° C.; s—Singlet; d—Doublet, t—Triplet; q—Quartet; m—Multiplet; dd—doublet of doublets; td—triplet of doublets; qd—quartet of doublets; ddd—doublet of doublet of doublets; dt—doublet of triplets; ddt—doublet of doublet of triplets; p—pentate; TLC—Thin Layer Chromatography; THF—Tetrahydrofuran; μ—Micron; μL—Micro litre and δ—Delta.

### EXPERIMENTAL

The present invention provides methods for the preparation of compound of formula (I) according to the procedures of the following examples, using appropriate materials. Those skilled in the art will understand that known variations of the conditions and processes of the following preparative procedures can be used to prepare these compounds. Moreover, by utilizing the procedures described in detail, one of ordinary skill in the art can prepare additional compounds of the present invention.

### HPLC

Purity analysis was performed on an Agilent HP1100 series system equipped with a diode array detector and using ChemStation software vB.04.03 using the method detailed below.

HPLC Parameters

| Parameter | Value | |
|---|---|---|
| Type of method | Reversed phase with gradient elution | |
| Sample Preparation | Diluent, acetonitrile/H₂O (55:45 v/v) | |
| Column | Sunfire C18, 250 × 4.6 mm, 5 μm | |
| Column Temperature | 45° C. | |
| Injection Volume | 10 μL | |
| Detector Wavelength, Bandwidth | UV at 210 nm | |
| Flow Rate | 1.0 mL/min | |
| Mobile Phase A | 10 mM KH₂PO₄ in H₂O (pH 6.20) | |
| Mobile Phase B | acetonitrile | |
| | Time (min) | % Mobile Phase A |
| Gradient Timetable | 0.0 | 65 |
| | 3.0 | 65 |
| | 30.0 | 40 |
| | 55.0 | 20 |
| | 65.0 | 20 |
| | 66.0 | 65 |
| | 75.0 | 65 |

In the preparation of compound of formula (I), it was observed that any specific stereoisomer of intermediate KRM-A underwent rapid racemization during isolating processes and resolving the desired enantiomer with high chiral purity had been challenging.

The inventors of the present invention have surprisingly discovered a solution to separate the two stereoisomers of the intermediate KRM-A, thus resulting in synthesis of the specific desired isomer of the compound of formula (I).

### Example-1: Preparation of Compound of Formula (I)

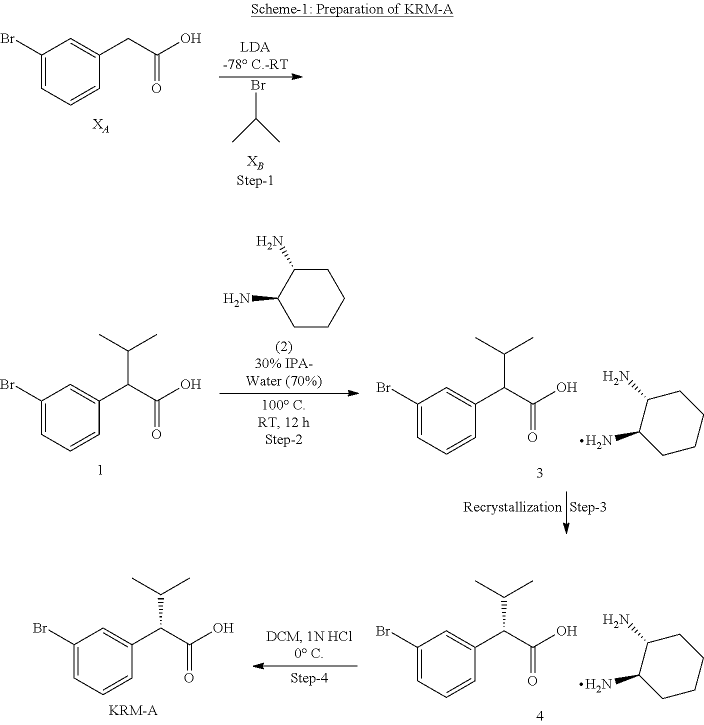

### Step-1: Preparation of 2-(3-bromophenyl)-3-methylbutanoic acid (1)

2M LDA (698 mL, 1.38mol) was added to a solution of 2-(3-bromophenyl) acetic acid (X_{A}, 150 g, 0.69 mol) in THF (700 mL) at −78° C.over a period of 30 min. The reaction mixture was stirred for 2 h at −78° C.followed by a drop wise addition of isopropyl bromide (X_{B}, 255 g, 2.07 mol) over a period of 30 min. The reaction mixture was stirred at room temperature overnight. Then, the reaction mixture was quenched with 1N HCl (pH 2) and the obtained product was extracted to ethyl acetate (500 mL×3). The combined organic layer was washed with water followed by brine solution. The organic layer was dried over anhydrous sodium sulfate and concentrated under reduced pressure to afford the crude compound which was purified by silica column by eluting with 0-10% ethyl acetate-hexane system to afford the title compound (150 g, 83% yield), HPLC purity-96%. The compound of formula (1) can also be prepared by the procedure described in CN110590747.

### Step-2: Preparation of Compound 3

2-(3-bromophenyl)-3-methylbutanoic acid (1, 510 g. 1.98 mol) was dissolved in 30% of IPA in water (10.2 L; 3.06 L of IPA-7.14 L of water) and (1R, 2R)-cyclohexane-1,2-diamine (2, 113 g. 0.9 mol) was added. The reaction mixture was stirred at room temperature for 10 min until the precipitation was observed, then was heated to 100° C.until the solution became clear and stirred at same temperature for another 30 min. The reaction mixture was allowed to slowly attain room temperature for 8-12 h. The obtained solid was filtered and washed with 500 mL of 30% IPA-water mixture and dried under vacuum to afford the compound 3 (620 g, wet).

Work up (for Chiral purity): Small portion (100 mg) of compound 3 was taken in DCM (2-3 mL) and was added 1N HCl (pH 2) at 0° C.until the clear solution was observed. The compound was extracted into DCM, dried over Na₂SO₄ and the solvent was evaporated to afford the title compound as white solid (20 mg). Chiral HPLC was recorded for this sample and 20.6% of undesired isomer was observed in chiral HPLC.

In order to improve the chiral purity of the title compound, the recrystallization method was performed as described below.

### Step-3: Recrystallization

The compound 3 (619.90 g) was taken in 30% of IPA in water (12.4 L), then the mixture was heated to 100° C.until the solution became clear and was stirred at same temperature for another 30 min. The reaction mixture was allowed to attain room temperature slowly for 8-12 h.

The obtained solid was filtered and washed with 500 mL 30% IPA-water and dried under vacuum to afford a desired compound (360 g, wet).

Work up for analysis (for Chiral purity): Small portion (100 mg) from above compound was taken in DCM (2-3 mL), was added 1N HCl (pH 2) at 0° C.until the clear solution was observed and the compound was extracted to DCM, dried over Na₂SO₄ and the solvent was evaporated to afford title compound as white solid (35 mg). Chiral HPLC was recorded for this sample and 10.3% of undesired isomer was observed in chiral HPLC.

The recrystallization method was repeated for three more times by using 30% of IPA in water as per the aforesaid procedure to get the purity of greater than 98.50% ee along with 0.27% other isomer to afford 286 g of compound 4.

### Step-4: Preparation of (S)-2-(3-bromophenyl)-3-methylbutanoic acid (KRM-A)

The compound 4 (286 g) was taken in DCM (1.3 L), then was added 1N HCl at 0° C. until the clear solution was observed, and the compound was extracted to DCM (500 mL×2). The organic layer was separated, washed with brine solution (500 mL) and dried over Na₂SO₄. The solvent was evaporated from the reaction mixture to afford title compound as white solid (148 g, 60% yield). Chiral HPLC: 98.50%

¹H NMR (400 MHZ, DMSO-d₆): δ 12.5 (s, 1H), 7.50-7.44 (m, 2H), 7.34-7.26 (m, 2H), 3.16 (d, 1H), 2.23-2.11 (m, 1H), 0.98 (d, 3H), 0.63 (d, 3H); Chiral HPLC: 98.50% retention time: 4.588 min.

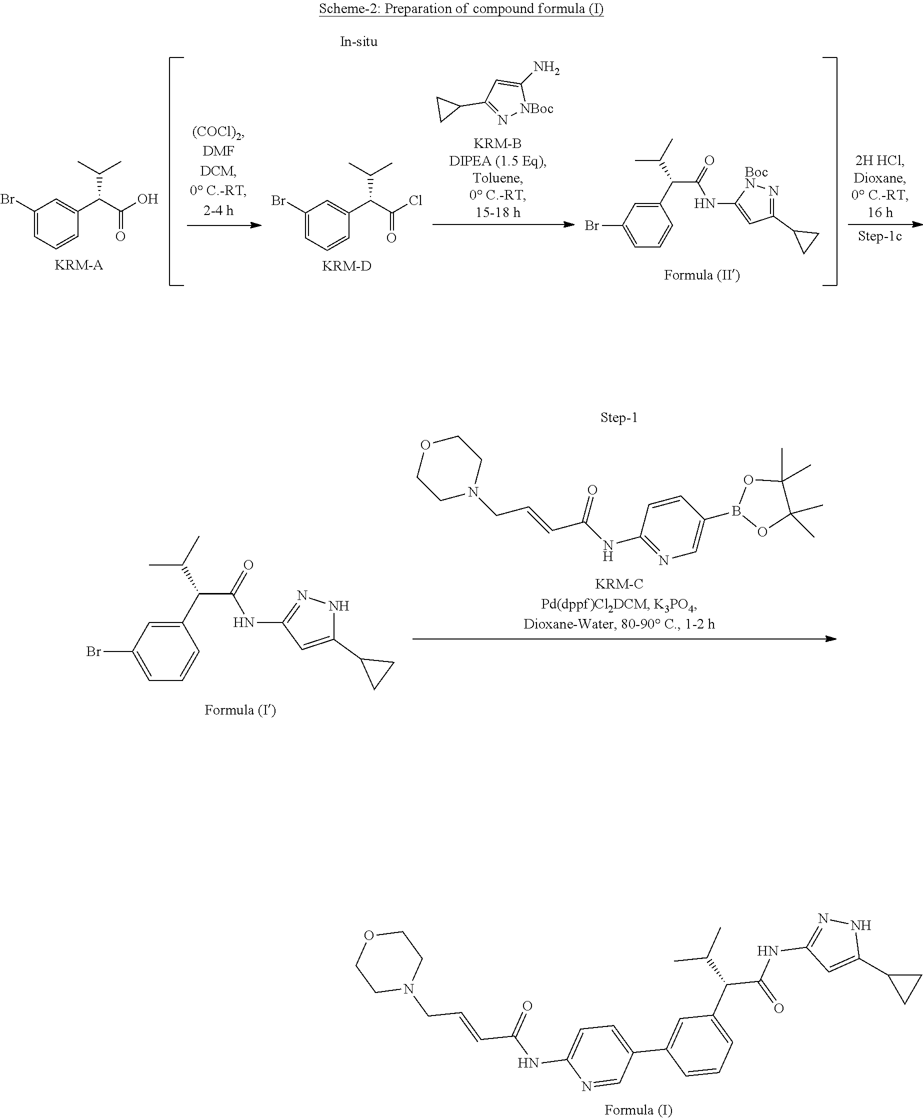

### Step-1: Synthesis of (S)-2-(3-bromophenyl)-N-(5-cyclopropyl-1H-pyrazol-3-yl)-3-methylbutanamide

### Step-1a: Preparation of KRM-D

To a stirred solution of KRM-A (100 g, 0.388 mol) in dry DCM (600 mL, 6 vol), a catalytic amount of DMF (10 mL) was added followed by oxalyl chloride (45 mL, 0.525 mol) dropwise at 0° C. over a period of 30 min. After completion of addition, the reaction mixture was stirred for 15 min at the same temperature. The reaction mixture was allowed to reach room temperature and stirred for 2 to 4 h. After completion of the reaction (reaction was monitored by TLC, acid chloride formation was checked by quenching an aliquot of reaction mixture with MeOH), the reaction mixture was concentrated under vacuum at 40° C.-45° C. to afford crude (S)-2-(3-bromophenyl)-3-methylbutanoyl chloride (KRM-D). The crude KRM-D was dissolved in toluene (500 mL) and used for next step.

### Step-1b: Preparation of Compound of Formula (II′)

(S)-2-(3-bromophenyl)-3-methylbutanoyl chloride in toluene was added slowly to a pre-cooled solution (0 to 5° C.) of tert-butyl 3-amino-5-cyclopropyl-1H-pyrazole-1-carboxylate (KRM-B, 95.5 g, 0.427 mol) and N,N-diisopropylethyl amine (100 mL, 0.583 mol) in toluene (1.2 L) at 0° C.for the period of 1-2 h. The reaction mixture was allowed to attain RT and stirred overnight. The reaction mixture was then cooled to 0-5° C. and washed with ice-cold 1.5N HCl (3×500 mL). The organic layer was washed with sodium bicarbonate solution (500 mL), brine solution (500 mL), dried over anhydrous Na₂SO₄, filtered and concentrated under vacuum at 45-50° C.to afford crude tert-butyl (S)-5-(2-(3-bromophenyl)-3-methylbutanamido)-3-cyclopropyl-1H-pyrazole-1-carboxylate (compound of formula (II′)) as light brown oil (˜180 g, LCMS: m/z=461.9 (M+H)⁺, HPLC: 80.80%, retention time: 15.89 min). The crude product was taken as such for next step without further purification.

### Step-1c: Preparation of Compound of Formula (I′)

To a suspension of tert-butyl (S)-5-(2-(3-bromophenyl)-3-methylbutanamido)-3-cyclopropyl-1H-pyrazole-1-carboxylate (180 g, 1.731 mol) in dioxanc (360 mL) was added 2N aqueous HCI (360 mL) at 0° C. The reaction mixture was stirred overnight at room temperature. After completion of the reaction, dioxane was concentrated, and the reaction mixture was diluted with water (500 mL) and basified with solid sodium bicarbonate (until pH-8). The obtained compound was extracted with DCM (700 mL×3). The combined organic layers were washed with water (300 mL), brine solution (300 mL), and dried over anhydrous Na₂SO₄. The organic layer was concentrated to get a crude (S)-2-(3-bromophenyl)-N-(5-cyclopropyl-1H-pyrazol-3-yl)-3-methylbutanamide (Compound of formula (I′)) as a semi solid. The crude was dissolved in toluene (500 mL) and the solution was stirred for 18 h. The obtained solid was filtered and washed with toluene (100 mL) and n-heptane (200 mL). The solid was further dried under vacuum at 45-50° C. for 6 h to afford a title compound (110 g, Yield: 78% over two steps). LCMS: m/z=362 (M+H)⁺, HPLC: 97.66%, retention time: 24.10 min

### Step-2: Preparation of (S,E)-N-(5-(3-(1-((5-cyclopropyl-1H-pyrazol-3-yl) amino)-3-methyl-1-oxobutan-2-yl)phenyl)pyridin-2-yl)-4-morpholinobut-2-enamide (Compound of formula (I))

To a degassed solution of (S)-2-(3-bromophenyl)-N-(5-cyclopropyl-1H-pyrazol-3-yl)-3-methylbutanamide (50 g, 0.138 mol) and (E)-4-morpholino-N-(5-(4,4,5,5-tetramethyl-1,3,2-dioxaborolan-2-yl)pyridin-2-yl)but-2-enamide (KRM-C, 56.6 g, 0.151 mol, 1.1 eq) (prepared according to the procedure described in WO2020202001) in 1,4-dioxane (500 mL, 10 vol) and water (100 mL, 2 vol) was added K₃PO₄ tribasic (73.2 g, 0.345 mol, 2.5 eq) at room temperature The reaction mass was stirred for 20 min with argon purging (degassing). Pd(dppf)Cl₂·DCM (3.38 g. 0.0042 mol, and 0.03eq) was added to the reaction mixture and the reaction mixture was heated to 90° C. for 1-2 h (The reaction was monitored by TLC using 10% methanol in DCM as solvent system).

After completion of the reaction, the reaction mass was cooled to room temperature and filtered through Celite® bed. The bed was washed with 1,4-dioxanc (200 mL) and the filtrate was concentrated to get crude compound. The crude compound was dissolved in 5% methanol in DCM (400 mL) and washed with water (200 mL×2). The aqueous layer was separated and extracted with DCM (100 mL×2). The combined organic layer was washed with brine solution, filtered and dried over sodium sulphate. The organic layer was concentrated under vacuum at 35-40° C.to get crude title compound (˜80 g).

The crude compound of formula (I), (80 g) was dissolved in 700 mL of ethyl acetate. The reaction mixture was cooled to 15° C. and 2N HCl was slowly added (until pH ˜1). The reaction mixture was then stirred at room temperature for 20 min and the layers were separated. The aqueous layer (containing the product) was washed with ethyl acetate (300 mL×3). The aqueous layer was cooled to 0° C. and adjusted the pH to ˜8 using 20% aqueous Na₂CO₃ solution. The product was extracted with 10% methanol in DCM (300 mL×3). The combined organic layer was washed with water (300 mL), dried over sodium sulphate and filtered. The filtrate was treated with activated charcoal (16 g, 20% w/w with respect to crude input of 80 g), then the reaction mixture was stirred overnight at room temperature and filtered through Celite® bed. The bed was washed with 5% methanol in DCM (˜20 vol, until absence of product by TLC). The filtrate was concentrated under vacuum at 35° C.-40° C. to afford compound of formula (I) (70 g, HPLC purity: 92.70%, retention time: 15.65 min).

Work-up for improved chiral purity: The above compound of formula (I) was dissolved in ethylacetate (˜30 vol, 2 L) and washed with aqueous citric acid (2 times, 400 mL×1 and 200 mL×1), aqucous NaHCO₃ solution (2%, 500 mL×1) and aqueous NaCl solution (10%, 500mL×1). The combined organic layer was dried over sodium sulphate and filtered. The filtrate was concentrated under vacuum at 35° C.-40° C. to afford compound of formula (I) (˜60 g).

¹H NMR (400 MHZ, DMSO-d₆): δ: 10.79 (s, 1H), 10.46 (s, 1H), 8.61 (d. 1H), 8.28 (d, 1H), 8.07-8.05 (m, 1H), 7.69 (s. 1H), 7.56 (d. 1H), 7.39 (m, 2H), 6.84-6.77 (m, 1H), 6.62 (s, 2H), 6.51 (d, 1H), 6.13 (s, 1H), 3.62-3.59 (m, 4H), 3.35 (d, 1H), 3.15-3.13 (m, 2H), 2.42-2.39 (m, 5H), 1.80-1.77 (m, 1H), 0.98 (d, 3H), 0.88-0.85 (m, 2H), 0.67 (d, 3H), 0.62-0.60 (m. 2H); LCMS: m/z=529.25-free base (M+H)⁺, HPLC: 98.98%, retention time: 15.40 min.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for preparing a compound of formula (I): 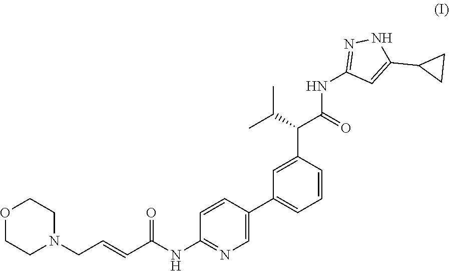 [SMILES: CC(C)[C@H](C(=O)Nc1cc(C2CC2)[nH]n1)c1cccc(-c2ccc(NC(=O)/C=C/CN3CCOCC3)nc2)c1]
wherein the method comprising reacting a compound of formula (I′″): 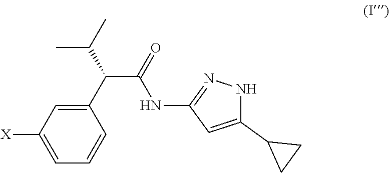
with a compound of formula (KRM-C1) 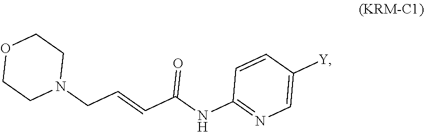
wherein X is Br, Cl or I; Y is —B(OH)₂ or 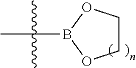
optionally substituted with 1, 2, 3 or 4 independently selected C₁₋₄ alkyl substituents, wherein the subscript n is 1 or 2.

2. The method of claim 1, wherein the reaction is carried out in the presence of a palladium catalyst.

3. The method of claim 1 or 2, wherein the compound of formula (I′″) is prepared by reacting a compound of formula (KRM-A1): 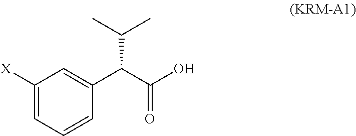
with a comound of formula (KRM-B): 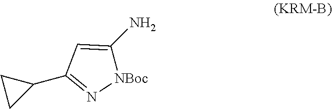
wherein X is Br, Cl or I.

4. The method of claim 3, wherein the compound of formula (KRM-A1) is prepared by a method comprising:
a) reacting a compound of formula (KRM-A2): 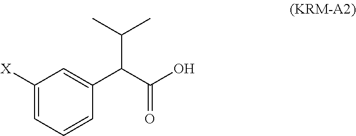
with (1R,2R)-cyclohexane-1,2-diamine to form a salt of formula: 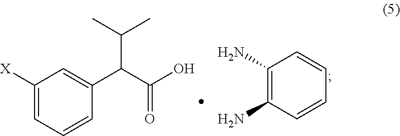
b) recrystallizing the salt (5) to obtain salt (6) of formula: 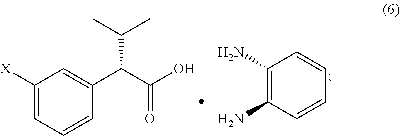
and
c) treating the salt of formula (6) with an acid to obtain the compound of formula (KRM-A1).

5. The method of claim 4, wherein the recrystallization is carried out in acetone, acetonitrile, methanol, isopropyl alcohol, isopropyl acetate, isobutanol, 1-pentanol, 1-propanol, ethanol, water, or a mixture thereof.

6. The method of claim 4 or 5, wherein the acid is HCl.

7. A method for preparing a compound of formula (I): 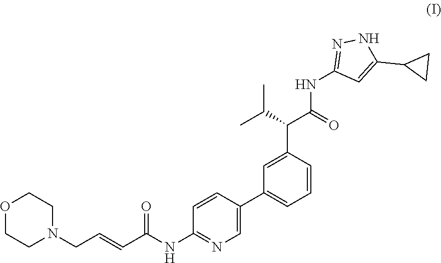 [SMILES: CC(C)[C@H](C(=O)Nc1cc(C2CC2)[nH]n1)c1cccc(-c2ccc(NC(=O)/C=C/CN3CCOCC3)nc2)c1]
wherein the method comprising:
i. reacting a compound of formula (1) 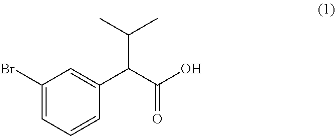
with a compound of formula (2) 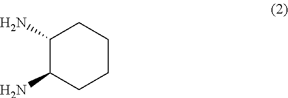
to afford a salt of a formula (3) 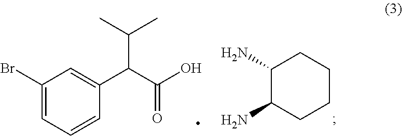
ii. recrystallizing the salt of formula (3) in a solvent (B) to obtain a salt of a formula (4) 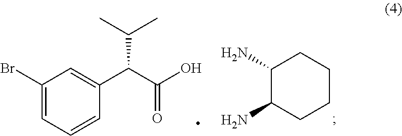
iii. treating a mixture comprising the salt of formula (4) and a solvent (C) with an acid to generate compound of formula (KRM-A) 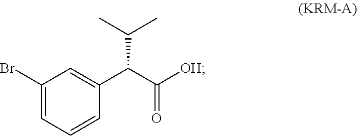
iv. reacting the compound of formula (KRM-A) with a compound of formula (KRM-B) 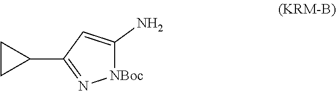 [SMILES: CC(C)(C)OC(=O)n1nc(C2CC2)cc1N]
to afford a compound of formula (I′): 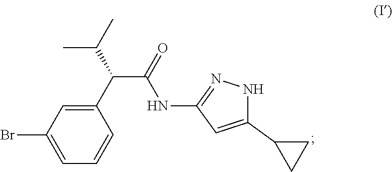 [SMILES: CC(C)[C@H](C(=O)Nc1cc(C2CC2)[nH]n1)c1cccc(Br)c1]
and
v. reacting the compound of formula (I′) with a compound of formula (KRM-C) 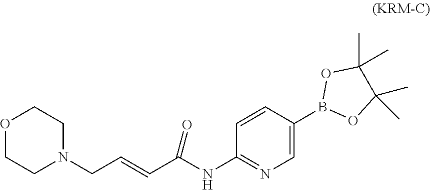 [SMILES: CC1(C)OB(c2ccc(NC(=O)/C=C/CN3CCOCC3)nc2)OC1(C)C]
to obtain the compound of formula (I).

8. The method of claim 7, wherein the step i) is carried out in the presence of the solvent (A) selected from acetone, dichloromethane, n-propyl acetate, acetonitrile, methanol, isopropyl alcohol, isopropyl acetate, isobutanol, 2-butanol, 1-butanol, n-butyl acetate, 1-pentanol, 1-propanol, chloroform, methyl acetate, isobutyl acetate, isobutanol, ethanol, water, or mixtures thereof.

9. The method of claim 8, wherein the solvent (A) is acetonitrile, isopropyl alcohol, isopropyl acetate, water or mixtures thereof.

10. The method of claim 7, wherein the step i) is carried out at a temperature between about 60° C.to about 100° C.

11. The method of claim 7, wherein the step ii) comprises:
a) providing a mixture comprising the compound of formula (3) and the solvent (B);
b) heating the mixture to form a solution; and
c) bringing the solution to supersaturation thereby causing the compound of formula (4) to precipitate out of the solution.

12. The method of claim 11, wherein the solvent (B) is acetone, acetonitrile, methanol, isopropyl alcohol, isopropyl acetate, isobutanol, 1-pentanol, 1-propanol, ethanol, water, or mixtures thereof.

13. The method of any one of claims 11 to 12, wherein the solvent (B) is isopropyl alcohol or water or any mixtures thereof.

14. The method of claim 11, wherein the step of bringing the solution to supersaturation comprises cooling the solution to ambient temperature or lower.

15. The method of claim 11, wherein the step of bringing the solution to supersaturation comprises maintaining a solution temperature above about 20° C.

16. The method of any one of claims 11 to 15, further comprising filtering the compound of formula (4) from the mixture comprising the compound of formula (4).

17. The method of claim 11, wherein the steps (a) to (c) are repeated at least three times.

18. The method of any one of claims 7 to 17, wherein the mixture comprising compound of formula (4) and a solvent (C) is a suspension.

19. The method of any one of claims 7 to 18, wherein the step iii) comprises cooling the mixture comprising compound of formula (4) and solvent (C).

20. The method of any one of claims 18 to 19, wherein the solvent (C) is acetone, dichloromethane, n-propyl acetate, acetonitrile, methanol, isopropyl acetate, isobutanol, 2-butanol, 1-butanol, n-butyl acetate, 1-pentanol, 1-propanol, chloroform, methyl acetate, isobutyl acetate, isobutanol or ethanol.

21. The method of any one of claims 7 to 20, wherein the acid is HCl.

22. The method of any one of claims 7 to 21, further comprising isolating the compound of formula (KRM-A) from the mixture.

23. The method of claim 22, wherein the mixture is a solution and isolating the compound of formula (KRM-A) comprises filtering the compound of formula (KRM-A) from the mixture.

24. The method of claim 7, wherein the compound of formula (I′) is prepared by reacting a compound of formula (II′): 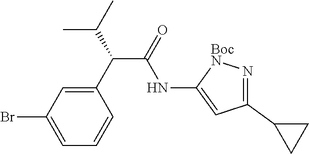 [SMILES: CC(C)[C@H](C(=O)Nc1cc(C2CC2)nn1C(=O)OC(C)(C)C)c1cccc(Br)c1]
with a deprotecting agent.

25. The method of claim 24, wherein the deprotecting agent is HCl.

26. The method of claim 24, wherein the compound of formula (II′) is prepared by i) reacting the compound of formula (KRM-A) with oxalyl chloride to generate a compound of formula 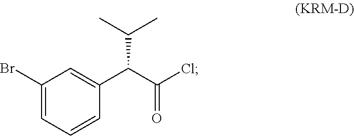
and ii) reacting the compound of formula (KRM-D) in situ with the compound of formula (KRM-B).

27. The method of claim 26, wherein the step ii) is carried out in the presence of a base.

28. The method of claim 27, wherein the base is N,N-diisopropylethylamine.

29. The method of any one of claims 7, 24 to 28, wherein the step v) is carried out in the presence of a palladium catalyst.

30. The method of claim 29, wherein the step v) is carried out in the presence of a solvent.

31. The method of claim 30, wherein the solvent is mixture of dioxane and water.

32. A compound of formula (III) 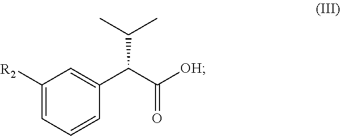
wherein
R² is halo, methylsulfonyloxy, p-tolylsulfonyloxy, phenylsulfonyloxy or trifluoromethylsulfonyloxy.

33. The compound of claim 32, wherein R² is halo.

34. The compound of any one of claims 32-33, having the structure:

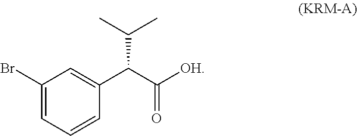

35. A compound of formula (IV): 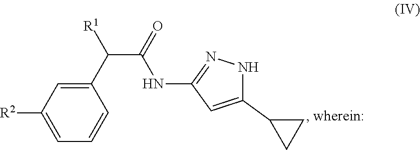
R¹ is C₁₋₆ alkyl; and R² is halo, methylsulfonyloxy, p-tolylsulfonyloxy, phenylsulfonyloxy or trifluoromethylsulfonyloxy.

36. The compound of claim 35, wherein R² is halo.

37. The compound of any one of claims 35 to 36, having the structure: 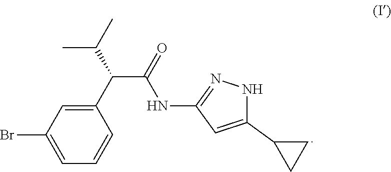 [SMILES: CC(C)[C@H](C(=O)Nc1cc(C2CC2)[nH]n1)c1cccc(Br)c1]
